(19) United States
(12) Patent Application Publication
(10) Pub. No.: US 2023/0191071 A1
(43) Pub. Date: Jun. 22, 2023

(54) COATED ANESTHETIC CONTAINER FOR AN ANESTHETIC DISPENSER AND MANUFACTURING PROCESS

(71) Applicant: Drägerwerk AG & Co. KGaA, Lübeck (DE)

(72) Inventors: Sven PASDZIOR, Lübeck (DE); Klaus RADOMSKI, Lübeck (DE); Sascha REINSCHMIEDT, Lübeck (DE)

(21) Appl. No.: 18/067,819

(22) Filed: Dec. 19, 2022

(30) Foreign Application Priority Data
Dec. 21, 2021 (DE) 10 2021 134 138.8

(51) Int. Cl. A61M 16/18

(52) U.S. Cl. CPC A61M 16/183

(57) ABSTRACT

A coated anesthetic container for an anesthetic dispenser includes an anesthetic tank, which is capable of receiving a liquid anesthetic (Nm), as well as a refill unit for refilling liquid anesthetic (Nm). The anesthetic tank includes a wall and a coating on the inner surface of the wall. A wall of the refill unit is connected in a fluid-tight manner to the wall of the anesthetic tank. A coating is applied at least to the inner surface of the wall of the anesthetic tank. This coating is made of an alloy of nickel and phosphorus. The nickel portion is in a range of 80 wt.% to 97 wt.%, and the phosphorus portion is in a range of 3 wt.% to 15 wt.%. A process is provided for manufacturing such an anesthetic container.

### CROSS REFERENCE TO RELATED APPLICATIONS

This application claims the benefit of priority under 35 U.S.C. § 119 of German Application 10 2021 134 138.8, filed Dec. 12, 2021, the entire contents of which are incorporated herein by reference.

### TECHNICAL FIELD

The present invention pertains to a coated anesthetic container for an anesthetic dispenser as well as to an anesthetic dispenser with such an anesthetic container and to a system for the mechanical ventilation and anesthetization of a patient with such an anesthetic dispenser. Furthermore, the present invention pertains to a process for manufacturing such an anesthetic container.

### BACKGROUND

In order to sedate or anesthetize a patient, usually a gas mixture, which comprises oxygen and at least one anesthetic, is fed to the patient. A patient-side coupling unit is arranged at or in the body of the patient. In many cases an anesthesia device carries out a sequence of ventilation strokes. In each ventilation stroke, a quantity of this gas mixture is delivered to the patient-side coupling unit.

In many cases, a delivery unit of the anesthesia device is in a fluid connection with an anesthetic vaporizer. This anesthetic vaporizer receives liquid anesthetic from the anesthetic container, vaporizes or evaporates this liquid anesthetic and produces gaseous anesthetic as a result. The anesthetic vaporizer often belongs to a gas mixture generator, and the gaseous anesthetic is mixed together with a carrier gas in a mixing chamber of the gas mixture generator and fed into the gas flow, which the delivery unit delivers to the patient-side coupling unit.

The liquid anesthetic is stored in an anesthetic container with an anesthetic tank. It is necessary from time to time to refill liquid anesthetic into the anesthetic tank. Such an anesthetic container is described, for example, in DE 10 2004 041 448 B3 and US 7,648,039 B2. The wall of the anesthetic tank is made of a material that contains aluminum.

### SUMMARY

A basic object of the present invention is to provide an anesthetic container to be used in an anesthetic dispenser, wherein the anesthetic container offers a higher reliability than prior-art anesthetic containers. In addition, the basic object of the present invention is to provide a process for manufacturing such an anesthetic container wherein the process is more reliable than prior-art manufacturing processes.

The object is accomplished by an anesthetic container having features described and by a manufacturing process having features described. Features and advantageous embodiments of the manufacturing process are also advantageous embodiments of the anesthetic container according to the present invention, where appropriate, and vice versa.

An anesthetic container according to the present invention is configured to be used as a part of an anesthetic dispenser. An anesthetic dispenser is defined as a unit with an anesthetic container, which stores a liquid anesthetic in the anesthetic container and generates and provides gaseous anesthetic with the use of the stored liquid anesthetic. The anesthetic container according to the present invention belongs to or is suitable for use in such an anesthetic dispenser. The anesthetic dispenser may comprise at least two anesthetic containers, especially for different anesthetics, wherein at least one container, preferably both containers, is/are configured and preferably manufactured according to the present invention.

The anesthetic container according to the present invention comprises an anesthetic tank. This anesthetic tank encloses an interior and is capable of receiving a liquid anesthetic in this interior. Furthermore, the anesthetic container comprises a refill unit, especially a neck or a refill opening. Liquid anesthetic can be refilled into the anesthetic tank through this refill unit. The refill unit can preferably be closed with a closure in a fluid-tight manner. The closure can be removed from the refill unit in order to refill liquid anesthetic and be put back on later.

Note: It is possible that a part of the liquid anesthetic vaporizes already during the refilling or later in the anesthetic tank. The remaining part of the anesthetic reaches the anesthetic tank in a liquid state during the refilling. However, at least one part of this anesthetic in the anesthetic tank also continues to be liquid later.

The anesthetic tank comprises a wall. A coating is applied to the inner surface of the wall of the anesthetic tank. This inner surface points toward the interior and thus toward the liquid anesthetic in the anesthetic tank. The coating is consequently located between the wall and the interior/liquid anesthetic. The coating is preferably configured such that the wall does not come directly into contact with the liquid anesthetic at any point, but rather the coating is ideally located in-between at any point.

The coating on the inner surface of the anesthetic tank is manufactured from an alloy. This alloy comprises nickel and phosphorus. The nickel portion (share) in the alloy is in a range of 80 wt.% to 97 wt.%, preferably in a range of 85 wt.% to 90 wt.%. The phosphorus portion (share) in the alloy is in a range of 3 wt.% to 15 wt.%, preferably in a range of 10 wt.% to 13 wt.%. Of course, the sum of the nickel portion and of the phosphorus portion is at most 100 wt.%.

The coating according to the present invention on the inner surface of the anesthetic tank reduces the risk that the material of the wall acts chemically on an anesthetic in the anesthetic tank. This chemical action could alter the anesthetic adversely. The coating prevents or at least reduces the risk that the wall comes into contact with the anesthetic and that an undesirable chemical action takes place on the anesthetic as a result.

According to the present invention, the coating between the wall and the interior comprises an alloy consisting of nickel and phosphorus. This alloy reacts chemically only to a relatively small extent with a liquid or even gaseous anesthetic. Therefore, the anesthetic tank is capable of storing liquid anesthetic, both over a longer period of time and at higher ambient temperatures as well, without the anesthetic being substantially chemically altered. This property is especially important when an anesthetic dispenser with an anesthetic container according to the present invention is kept in reserve and is used only if a different anesthetic dispenser is no longer capable of providing a gaseous anesthetic. It is especially necessary in many cases, in particular during an ongoing anesthetization of a patient, to switch over from an anesthetic dispenser with an empty anesthetic tank to another anesthetic dispenser, which was kept in reserve beforehand, possibly for a longer period of time.

Moreover, the coating on the inner surface reduces the risk that a liquid anesthetic in the anesthetic tank acts on the wall chemically. This could lead to a leak in the wall, which in turn often has the undesirable consequence that the anesthetic is released into the surrounding area.

The alloy composed of nickel and phosphorus usually has a sufficient mechanical resistance. The coating is hence preserved in many cases even if the anesthetic container is exposed to mechanical actions from outside, for example, impacts or shocks, and/or changing ambient temperatures.

The wall of the anesthetic tank may have a relatively complicated geometry and, in particular, it may comprise corners and/or edges with a small radius of curvature and/or undercuts and/or indentations. A uniform coating of the inner surface of the anesthetic tank can be obtained in many cases thanks to the present invention even in case of a relatively complicated geometry. “Uniform” means especially that all areas of the inner surface are covered with the coating and ideally no contact surface at all occurs, in which an anesthetic in the anesthetic tank has a direct contact with the wall. In practice, it is possible in many cases to achieve that the contact surface remaining after the coating has at most 5%, in many cases even at most 1%, of the entire area of the inner surface.

It is possible in many cases to apply the coating to the wall such that the coating has a desired and a relatively uniform layer thickness, i.e., the layer thickness does not vary from a desired mean layer thickness value by more than ± 5 µm. This coating with a uniform layer thickness can in many cases be obtained even if the wall of the anesthetic tank has the just mentioned relatively complicated geometry. Moreover, a coating according to the present invention often leads to an especially smooth surface. A smooth surface reduces the effective area and further reduces the risk that the anesthetic in the anesthetic tank comes into direct contact with the wall.

The coating preferably has a layer thickness in a range of 0.5 µm to 80 µm, especially preferably in a range of 10 µm to 20 µm.

According to the present invention, the phosphorus portion in the alloy is in a range of 3 wt.% to 15 w.%. This portion is preferably in a range of 10 wt.% to 13 wt.%. The coating has an amorphous structure in case of a phosphorus portion above 10 wt.%,. This reduces the risk that inhomogeneities, for example, grain boundaries or separated phases, will develop in the coating. Such inhomogeneities can reduce the mechanical resistance of the coating. Furthermore, because of the amorphous structure obtained, a phosphorus portion above 10 wt.%. reduces the risk that crystals and/or large pores develop in the coating. Both crystals and pores may lead to gaps in the coating and as a result cause a larger quantity of the anesthetic to come into direct contact with the wall.

During the refilling, liquid anesthetic usually flows through the refill unit into the anesthetic tank. In one embodiment, the refill unit also comprises a wall, especially if the refill unit is configured as a neck. The wall of the refill unit is connected to the wall of the anesthetic tank in a fluid-tight manner. It is possible that the wall of the anesthetic tank and the wall of the refill unit form a single part. The two walls are preferably manufactured from a rigid material.

It is possible that a coating is likewise applied to the inner surface of the wall of the refill unit. It is also possible that a coating is likewise applied to the outer surface of the wall of the anesthetic tank and/or of the refill unit.

In one embodiment, the inner surface of the wall of the refill unit is not coated. Such a coating is not necessary in some cases, especially if the level of the liquid anesthetic in the anesthetic tank remains below the refill unit and therefore the refill unit comes into contact with anesthetic only during the refilling. In another embodiment, the inner surface of the wall of the refill unit and the inner surface of the wall of the anesthetic tank are provided with a coating made of the same material. The entire inner surface of the wall of the anesthetic container is especially preferably covered by a continuous coating except for the inner surface of an optional closure for the refill unit and except for an optional visual inspection unit, which will be described below.

It is preferably possible to visually determine from outside the upper level and as a result the fill level of liquid anesthetic in the anesthetic tank. A user or even a camera in conjunction with an image analysis unit can determine the fill level from outside. In order to make this determination possible, the anesthetic container additionally comprises a visual inspection unit. This visual inspection unit is transparent and makes possible a view of the interior of the anesthetic tank from outside. The visual inspection unit may comprise an inspection glass in the wall of the anesthetic tank. It is also possible that the visual inspection unit comprises an inspection tube made of a transparent material. This inspection tube is in at least one fluid connection with the anesthetic tank such that the upper level of liquid anesthetic in the inspection tube is on the same horizontal plane as the upper level of liquid anesthetic in the anesthetic tank.

The visual inspection unit is preferably made of a transparent material, which has a quartz (silicon dioxide, SiO₂) portion of at least 70 wt.%. The quartz portion is preferably at least 80 wt.%, especially preferably at least 90 wt.%, and especially at least 99 wt.%.

If the quartz portion is high enough, then the material of the visual inspection unit is in many cases not acted on by liquid anesthetic in a relevant way. The inventors found in internal experiments that in case of a quartz portion of at least 80 wt.%, often neither a noteworthy action of the liquid anesthetic on the visual inspection unit nor an action of the material of the visual inspection unit on the liquid anesthetic occurs.

In one embodiment, a transparent coating is applied to the inner surface of the visual inspection unit. This coating is located between the visual inspection unit, on the one hand, and the interior and therefore the liquid anesthetic in the anesthetic tank, on the other hand. Such a coating is especially necessary or at least meaningful in many cases when the quartz portion is below 80 wt.%. This transparent coating is preferably manufactured from a plastic. This plastic especially preferably comprises at least one of the following materials: Parylene, a polymer, preferably an epoxy phenolic polymer, polytetrafluoroethylene (PTFE), polyolefin.

Both the wall of the anesthetic tank and the optional wall of the refill unit are preferably each manufactured from at least one solid material. It is possible that the two walls are manufactured from the same material. It is also possible that different materials are used. It is also possible that the wall of the anesthetic tank is manufactured from at least two different materials, especially if the wall comprises two layers.

In one configuration, the material, from which the wall of the anesthetic tank is manufactured, comprises at least one metal alloy. The aluminum portion in this metal alloy is at least 80 wt.%, preferably at least 90 wt.%, especially preferably at least 95 wt.%, according to one embodiment of this configuration. The material for the optional wall of the refill unit also preferably comprises at least one such metal alloy.

A wall with a high aluminum portion is relatively resistant to corrosion and in many cases also relatively resistant to a liquid anesthetic. Moreover, aluminum has a lower specific weight than many other metals. In addition, a blank made of a material having a high portion can frequently be more easily molded into a desired shape than a blank made of a different material, especially by means of a die-casting or extrusion process.

In another embodiment, the material, from which the wall of the anesthetic tank is manufactured, comprises at least one plastic. Plastic usually cannot corrode and often has a relatively low weight and in many cases a casting process by means of a mold can be used to manufacture the wall. A wall with a complicated geometry can frequently be manufactured more easily by means of a casting process than with a different process. The plastic preferably comprises at least one of the following materials: A polyamide, a polyphenylene sulfide, a polyether ether ketone (PEEK).

A combination of these two embodiments is possible. The wall comprises two layers. The one layer comprises a metal alloy, and the other layer comprises a plastic. The coating with nickel and phosphorus according to the present invention is applied to the inner surface of the inner wall. The layer with the metal alloy is preferably located between the coating according to the present invention and the layer with the plastic. This arrangement further reduces the risk of corrosion.

The present invention further pertains to a use of the anesthetic container according to the present invention as a part of an anesthetic dispenser as well as to an anesthetic dispenser with an anesthetic container according to the present invention. The anesthetic dispenser comprises the anesthetic container according to the present invention and, furthermore, a feed device and an anesthetic vaporizer. The feed device is at least from time to time in a fluid connection with the anesthetic container according to the present invention, so that liquid anesthetic can flow from the anesthetic container to the feed device. It is possible that this fluid connection is interrupted from time to time, for example, by means of an actuated valve or another final control element. The feed device is capable of feeding, for example, injecting or filling in, received liquid anesthetic into a chamber of the anesthetic vaporizer. The anesthetic vaporizer is capable of generating gaseous anesthetic in this chamber from the liquid anesthetic, for example, by heating and/or evaporating.

The present invention further pertains to a gas mixture generator
with an anesthetic dispenser, which is configured as just described and hence comprises an anesthetic container according to the present invention, and
with a gas mixer.

The gas mixer is at least from time to time in a fluid connection with the anesthetic dispenser, so that liquid anesthetic can flow from the anesthetic dispenser to the gas mixer. In addition, the gas mixer is at least from time to time in a fluid connection with a source for a carrier gas, wherein this carrier gas comprises oxygen. The gas mixer is capable of generating from the received carrier gas and from the received gaseous anesthetic a gas mixture, which comprises oxygen and liquid anesthetic. The gas mixer preferably mixes the gaseous anesthetic together with the carrier gas, so that the gas mixture is ideally homogeneous over its extension, i.e., it has the same anesthetic portion all over.

The present invention further pertains to a system for the mechanical ventilation of a patient. The patient is connected to a patient-side coupling unit or can be connected to such a patient-side coupling unit. The patient-side coupling unit preferably comprises a breathing mask or a tube or a catheter. A fluid connection is established or can be established between the ventilation system and the patient-side coupling unit at least from time to time.

The ventilation system comprises a gas mixture generator according to the present invention and an anesthetic container according to the present invention as well as a fluid delivery unit, for example, a pump or a piston and cylinder unit or even a ventilation bag to be actuated manually. The gas mixture generator is capable of generating a gas mixture comprising oxygen and at least one gaseous anesthetic. The fluid delivery unit is capable of delivering the gas mixture through the fluid connection to the patient-side coupling unit. A ventilation circuit is preferably established, i.e., exhaled breathing air can flow through the patient-side coupling unit back to the ventilation system. The ventilation circuit reduces the risk that a gaseous anesthetic will be released into the surrounding area or into a stationary infrastructure of a hospital.

In one embodiment, the ventilation system comprises a ventilator. This ventilator carries out a sequence of ventilation strokes and delivers in each ventilation stroke a respective quantity of the gas mixture containing oxygen and anesthetic to the patient-side coupling unit. The gas mixture generator may be a part of this ventilator.

Different processes are possible for manufacturing an anesthetic container according to the present invention. The wall of the anesthetic tank is preferably manufactured first. In one embodiment, a part is first manufactured, which part the wall of the anesthetic tank and the wall of the refill unit, wherein the two walls are preferably connected to one another permanently and in a fluid-tight manner.

In one embodiment, the coating is applied, for example, sprayed, onto the wall from the inside. In a preferred embodiment, by contrast, the wall of the anesthetic tank or the part with the two walls is moved relative to a dipping bath, especially lowered or inserted or dipped, into a dipping bath. The dipping bath provides a liquid which contains nickel and phosphorus. This liquid preferably fully encloses the wall or the part after the relative movement. The pH value of the liquid that is provided determines the phosphorus portion of the coating to be manufactured. Hence, a pH value for this liquid is derived and predefined beforehand as a function of a desired range for the phosphorus portion. Preferably the liquid contains sodium hydrogen phosphate (NaH₂PO₂) and a nickel sulfate, for example, NiSO₄, as well as at least one suitable solvent.

The coating according to the present invention is formed in the dipping bath on both sides of the wall, wherein ions are deposited on the wall and wherein the coating expands slowly. A lower threshold is preferably predefined for the required layer thickness. The wall of the part with the two walls is left in the dipping bath at least until the actual layer thickness of the coating has reached the required lower threshold.

The embodiment with the dipping bath leads in many cases to a uniform layer thickness over the entire extension of the wall or even of the entire part being obtained. Even when the wall has a relatively complicated geometry, a coating according to the present invention generated in a dipping bath frequently covers the entire inner surface of the wall. The residence time of the part in the dipping bath determines the layer thickness obtained. In addition, the use of a dipping bath often requires a manufacturing device, which has only relatively few moving parts. It is essentially only necessary to provide the dipping bath, to clean the part to be coated beforehand, to move it relative to the dipping bath, especially to lower it into the dipping bath and to remove it again from the dipping bath later.

It is possible to use a galvanic process or an anodizing process to form the coating according to the present invention in the dipping bath. In one embodiment, the coating in the dipping bath is carried out according to an electroless (chemical) nickel plating process. An electroless nickel plating process does not require a current to be applied to a part to be coated. Therefore, a wall to be coated may also be manufactured from a material, which is not electrically conductive or only slightly electrically conductive, for example, from a rigid plastic. Furthermore, an energy source and electric energy are not needed.

The present invention will be described below on the basis of exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a system for the mechanical ventilation of a patient;

FIG. 2 is a schematic view showing a single anesthetic dispenser;

FIG. 3 is a schematic view showing an anesthetic tank in a cross-sectional view; and

FIG. 4 is a schematic view showing a front view of the anesthetic tank from FIG. 3 with an inspection tube.

### DESCRIPTION OF PREFERRED EMBODIMENTS

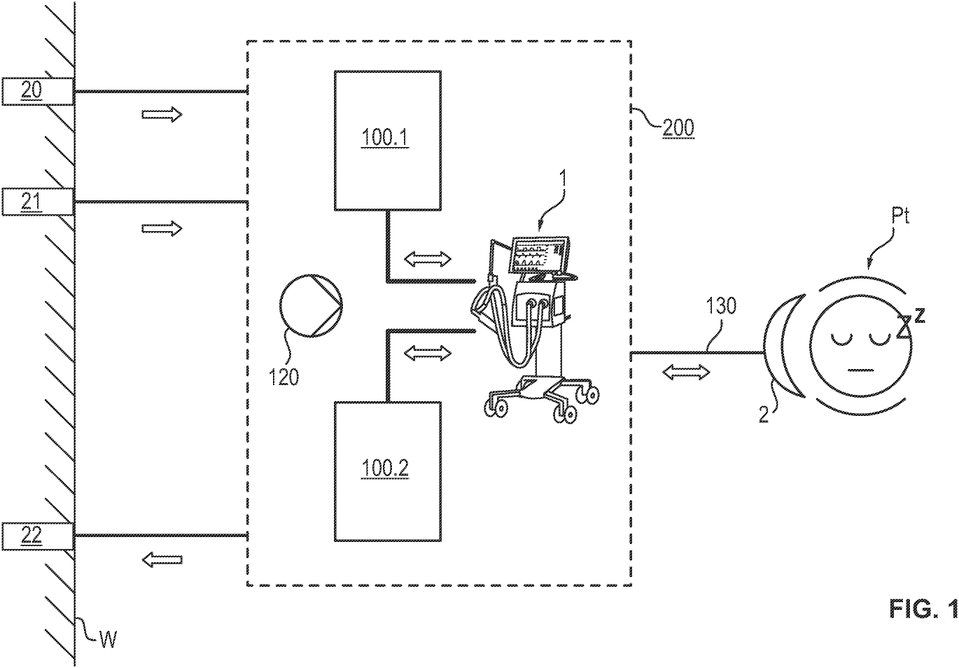

FIG. 1 schematically shows a system 200 for the mechanical ventilation of a patient Pt. The patient Pt is connected to a patient-side coupling unit 2, for example, to a breathing mask on the face or to a tube or to a catheter in the body of the patient Pt.

The ventilation system 200 carries out a sequence of ventilation strokes. During each ventilation stroke a ventilation gas mixture comprising oxygen and at least one anesthetic is delivered through a fluid connection 130 to the patient-side coupling unit 2. Because of the anesthetic in the gas mixture being fed, the patient Pt is sedated or even anesthetized. The air exhaled by the patient Pt is sent back to the ventilation system again, so that no anesthetic is released into the surrounding area. As a result, a ventilation circuit is established between the ventilation system 200 and the patient-side coupling unit 2. A fluid delivery unit in the form of a pump 120 maintains a gas flow in this ventilation circuit.

The ventilation system 200 comprises an anesthesia device 1, which carries out the ventilation strokes, and two gas mixture generators 100.1 and 100.2. The two gas mixture generators 100.1, 100.2 are connected to the anesthesia device 1 in a detachable manner. The pump 120 belongs to the anesthesia device 1. Each gas mixture generator 100.1, 100.2 is supplied with a carrier gas comprising oxygen from the anesthesia device 1 and generates an anesthetization gas mixture comprising the carrier gas and at least one anesthetic. The anesthesia device 1 generates the ventilation gas mixture with the use of the anesthetization gas mixture and delivers the ventilation gas mixture to the patient-side coupling unit 2. The oxygen portion content in the ventilation gas mixture may be the same as the oxygen portion in breathing air. It is possible that the anesthetization gas mixture is used as the ventilation gas mixture. The anesthesia device 1 optionally increases the oxygen portion in the ventilation gas mixture.

A gas mixture generator 100.1 or 100.2 is usually used to sedate or to anesthetize a patient Pt. The other gas mixture generator 100.2 or 100.1 is likewise connected to the anesthesia device 1 and is currently not used, but is available to be used immediately as needed. As a rule, it is possible to switch over rapidly from the one gas mixture generator 100.1 to the other gas mixture generator 100.2, without interrupting the anesthetization of the patient Pt.

FIG. 1 further shows a supply port 20 for the carrier gas and a supply port 21 for compressed air, wherein both supply ports 20, 21 are arranged in a wall W and belong to a stationary supply system. In the ventilation circuit, excess gas is generated, which is sent to a disposal port 22 in the wall W and is received there.

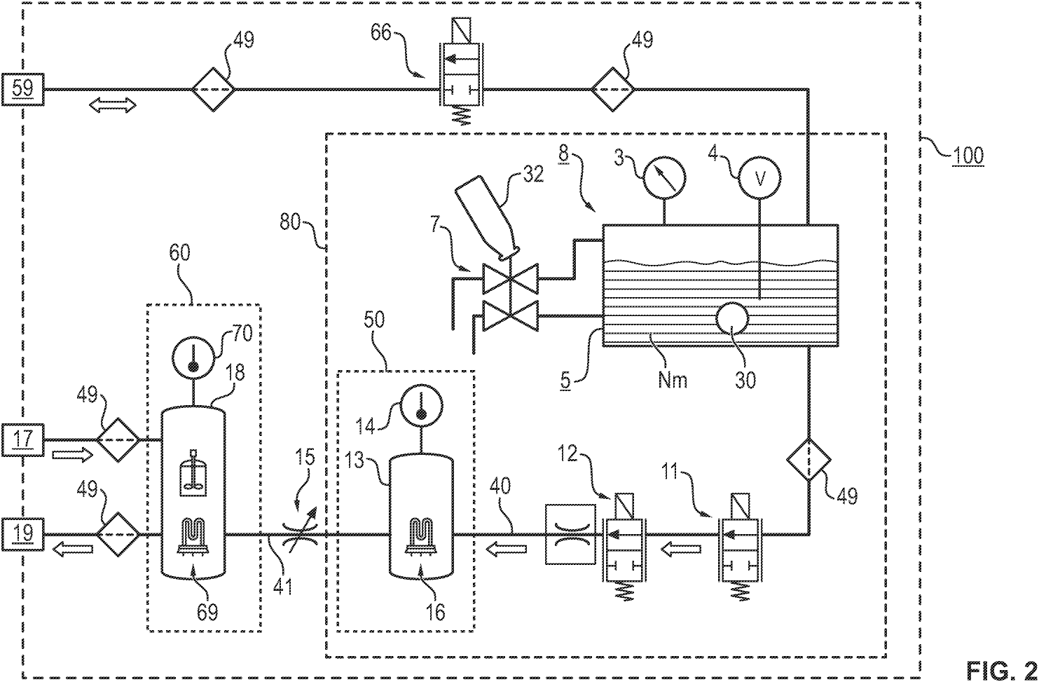

FIG. 2 schematically shows a single gas mixture generator 100. This gas mixture generator comprises an anesthetic dispenser 80 and a gas mixer 60. The carrier gas is fed to the gas mixer 60 via an inlet 17. The anesthetic dispenser 80 generates gaseous anesthetic, which is likewise fed to the gas mixer 60. The gas mixer 60 generates the anesthetization gas mixture from the gaseous anesthetic and the carrier gas. The anesthetization gas mixture generated is then discharged via an outlet 19 and delivered to the patient-side coupling unit 2, cf. FIG. 1.

Liquid anesthetic Nm is stored in an anesthetic tank 5 of an anesthetic container 8 of the anesthetic dispenser 80. It is possible that the anesthetic dispenser 80 comprises an additional anesthetic tank (not shown), wherein this additional anesthetic tank is in a fluid connection with the anesthetic tank 5 and hence does not necessarily comprise a closable refill unit of its own. This fluid connection acts as the refill unit of the additional anesthetic tank.

An inspection glass 30 is inserted into a wall of the anesthetic tank 5. A user or even a camera can visually determine from outside through the inspection glass 30 the current fill level of the liquid anesthetic in the anesthetic tank 5. A protective layer, which protects the inspection glass 30 from mechanical damage from outside, is located outside on the inspection glass 30 in one embodiment. A fill level sensor 4 measures a value indicative of the current fill level of the liquid anesthetic Nm in the anesthetic tank 5.

Liquid anesthetic Nm can be refilled through a closable neck 7. The neck 7 acts as the refill unit of the anesthetic tank 5 and comprises an adapter, onto which a container can be placed in a fluid-tight manner for refilling liquid anesthetic. A cylinder 32 containing liquid anesthetic is shown as an example as a refill container for refilling. The cylinder 32 can be placed onto an adapter at the neck 7 such that a fluid-tight connection is established and the liquid anesthetic can flow from the cylinder 32 through the neck 7 downwards into the anesthetic tank 5. The neck 7 is fastened to a side wall of the anesthetic tank 5 in the embodiment being shown. It may also be arranged in the cover 10 of the anesthetic tank 5.

A gas mixture comprising anesthetic is present in the anesthetic tank 5 above the level of the liquid anesthetic. The boiling point of some frequently used anesthetics is below 40° C. Hence, an overpressure compared to the ambient pressure especially develops in the interior of the anesthetic container 8. In the exemplary embodiment, the anesthetic container 8 is configured such that it can withstand an overpressure up to a structure-related overpressure threshold. This overpressure threshold is in a range of 1 bar to 50 bar, and preferably in a range of 1 bar to 20 bar.

A pressure sensor 3 measures a value indicative of the pressure of this gas mixture. The anesthetic tank 5 is connected to a port 59 via a line. The pressure in the anesthetic tank 5 can be altered by means of a proportional valve 66. A signal-processing control device, not shown, preferably receives measured values from the pressure sensor 3 and actuates the proportional valve 66 as a function of the measured pressure in the anesthetic tank 5. The control device actuates the proportional valve 66 with the control target that the actual pressure in the anesthetic tank 5 follows a predefined pressure time curve.

Liquid anesthetic Nm flows through a vaporizer feed line 40 to a vaporizer chamber 13 of an anesthetic vaporizer 50. An actuatable proportional valve 11 as well as a feed device in the form of an actuatable injection valve 12 are arranged in this line 40. The control device actuates the proportional valve 11 and as a result controls the volume flow of liquid anesthetic Nm through the line 40. The injection valve 12 injects liquid anesthetic Nm into the vaporizer chamber 13 of the anesthetic vaporizer 50. An actuatable heater 16 contributes to vaporizing the liquid anesthetic Nm in the vaporizer chamber 13. A temperature sensor 14 measures a value indicative of the temperature in the vaporizer chamber 13.

The gaseous anesthetic flows from the vaporizer chamber 13 through a mixer feed line 41 with a pneumatic resistance 15, which is preferably actuatable, into a mixing chamber 18 of the gas mixer 60. The gaseous anesthetic is mixed together with a carrier gas in this mixing chamber 18. An actuatable heater 69 is capable of heating the gas mixture in the mixing chamber 18. A temperature sensor 70 measures a value indicative of the temperature in the mixing chamber 18.

In addition, a plurality of filters 49 are shown in FIG. 2.

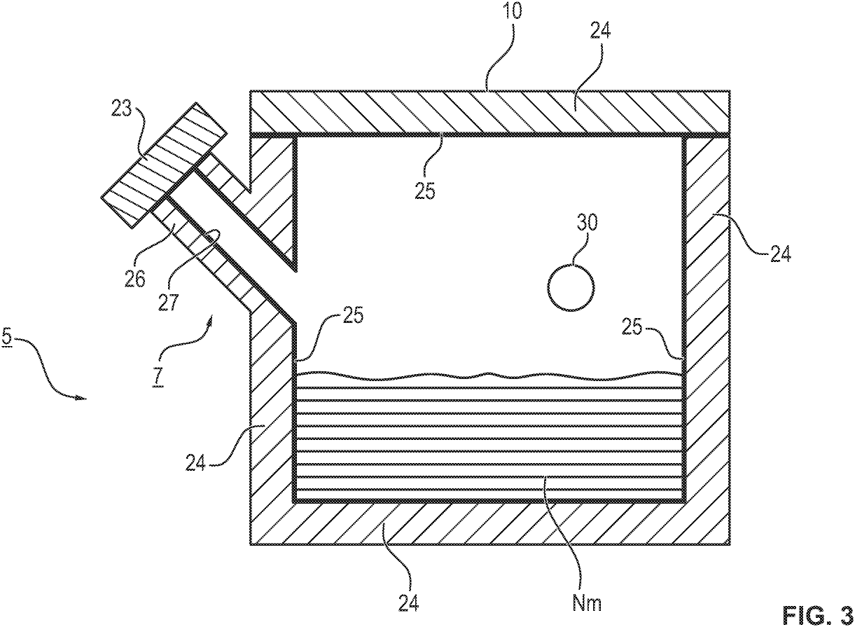

FIG. 3 schematically shows a cross section through the anesthetic container 8, wherein the anesthetic container 8 comprises the anesthetic tank 5, the neck 7, the closure 23 and the inspection glass 30. The geometry of the anesthetic container 8 is shown in a simplified manner. A tub of the anesthetic tank 5 is closed in a fluid-tight manner by a cover 10. During the regular operation, this cover 10 remains on the tub of the anesthetic tank 5. The cover may be opened, e.g., for the purpose of maintenance. The tub and the cover 10 are intended by “the anesthetic tank” below. The neck 7 is closed by a closure 23 in a fluid-tight manner. The closure 23 can be removed from the neck 7, especially in order to refill liquid anesthetic Nm into the anesthetic tank 5, without having to remove the cover 10.

The anesthetic tank 5 comprises a wall 24. This wall 24 is formed by the tub and by the cover 10 in the embodiment being shown. The neck 7 comprises a wall 26. The respective wall thickness of the wall 24, 26 is set such that the anesthetic container 8 can withstand an overpressure in its interior up to the above-mentioned overpressure threshold. The wall thickness is preferably in a range of 4 mm to 30 mm.

In one embodiment, the wall 24, 26 of the anesthetic container 8 is manufactured as one part. In another embodiment, two halves of the wall 24, 26 are manufactured separately from one another. These two halves are subsequently connected to one another by laser welding or by another joining technique. A process for manufacturing the wall 24, 26 of the anesthetic container 8 in such a manner is described in DE 10 2004 041 448 B3 and US 7,648,039 B2.

Different processes are possible for how the wall 24, 26 or even the two halves are manufactured. It is possible to manufacture the wall 24, 26 from a liquid material by means of a casting process, and preferably by means of die-casting. It is also possible to manufacture the wall 24, 26 from at least one metal sheet using an extrusion process. If an extrusion or casting process is used, then the wall thickness is preferably in a range of 4 mm to 23 mm. In another embodiment, the wall 24, 26 is manufactured by milling. The wall thickness may then be above 23 mm.

At least two of these processes can also be combined with one another. For example, some parts, for example, connection pieces between the neck 7 and the anesthetic tank 5, are manufactured by milling and the remaining parts are manufactured by die-casting or extrusion. The milled parts are subsequently connected to the remaining parts by means of laser welding or by means of another joined connection. It is possible that the parts of the wall 24, 26, which are manufactured using extrusion or casting, have a lower wall thickness than the parts that are manufactured by milling.

The walls 24 and 26 are preferably manufactured from the same material. It is possible that one material is used for die-casting or for extrusion and a different material is used for milling.

Different materials for the wall 24, 26 are possible. In a preferred embodiment, the material is a metal alloy, which has an aluminum (Al) portion of at least 80%, preferably of at least 90%, and especially preferably of 95%. Because the aluminum portion is at least 80%, the wall 24, 26 has a relatively low weight and can be molded into a desired shape relatively easily. Aluminum has, in addition, a sufficiently high corrosion resistance.

An aluminum alloy according to EN AW-6063 is especially preferably used as the material for the extrusion or die-casting. This material for the wall 24 has a magnesium (Mg) portion in a range of 0.45 wt.% to 0.9 wt.%, a silicon (Si) portion in a range of 0.2 wt.% to 0.6 wt.%, an iron (Fe) portion of 0.35 wt.% as well as additional elements with lower contents. An aluminum alloy according to EN AW-5083 is especially preferably used for the milling. The magnesium (Mg) portion is in a range of 4 wt.% to 4.9 wt.%, the manganese (Mn) portion is in a range of 0.4 wt.% to 1 wt.%, and the silicon (Si) portion and the iron (Fe) portion are each 0.4 wt.%.

The metal alloy may also have a magnesium (Mg) portion of at least 80 wt.%, and preferably of at least 90 wt.%. This alloy preferably contains an aluminum portion, which is in a range of 6 wt.% to 12 wt.%, as well as a zinc portion and a manganese portion, which are each below 1 wt.%. The metal alloy may also have a brass portion of at least 80 wt.%, preferably of at least 90 wt.%. As is known, brass is an alloy containing at least 50 wt.% copper (Cu) and at most 40 wt.% zinc (Zn).

It is also possible to use a plastic instead of a metal alloy. When the wall 24, 26 is manufactured from plastic and not from a metal alloy, then it is not magnetic and also cannot be magnetized. In addition, it is then not able to corrode and has in many cases a lower weight than a wall 24, 26 made of a metal alloy. A wall 24, 26 made of plastic is preferably manufactured by means of a casting process, and especially preferably by means of die-casting. At least one of the following plastics is preferably used:
a polyamide,
a polyphenylene sulfide (PPS) or
a polyether ether ketone (PEEK, also called PEAK).

It is desired that the wall 24, 26 be continuously made of a homogeneous material. However, in practice, pores may develop in the wall 24, 26. The wall 24, 26 is preferably manufactured such that the maximum diameter of a pore is at most 30 µm.

In one embodiment, the closure 23 is manufactured from a flexible plastic. As a result, the closure 23 fills out the entire cross-sectional area of the neck 7, on the one hand, and, on the other hand, can be compressed to pull the closure 23 from the neck 7, or it is compressed when the closure 23 is pulled from the neck 7. The material, from which the closure 23 is manufactured, preferably comprises a polyphenylene sulfide (PPS), especially preferably a polyphenylene sulfide reinforced with glass fiber. The closure 23 may also comprise a rigid part with an external thread, wherein the external thread meshes with an internal thread of the neck 7. The rigid closure 23 preferably comprises, furthermore, a seal.

The anesthetic tank 5 is capable of receiving a liquid anesthetic. The same anesthetic tank 5 is able to receive different anesthetics one after the other. During refilling, liquid anesthetic Nm flows through the neck 7 into the anesthetic tank 5. What requirements result from the anesthetic tank 5 receiving a liquid anesthetic and how these requirements are met according to the present invention will be explained below.

A frequently used anesthetic has become known by the name sevoflurane. Sevoflurane has the chemical empirical formula (CF3)₂CHOCHF₂ and the chemical name 1,1,1,3,3,3-hexafluoro-2-fluoromethoxy propane. Other frequently used anesthetics have the names isoflurane and desflurane.

It is known that liquid anesthetics, for example, the anesthetics just mentioned, are chemically aggressive. Hence, only materials with a sufficient chemical resistance to liquid anesthetics may be considered to be materials that come into contact with liquid anesthetics. The inventors have, in addition, found in internal experiments that a wall 24, 26 with a high aluminum portion can easily be manufactured, but has an undesirable action on a liquid anesthetic Nm in the anesthetic tank 5 under unfavorable conditions, especially when one of the anesthetics just mentioned is used. In particular, the metal alloy of the wall 24 may chemically alter the anesthetic Nm in the anesthetic tank 5 or reduce the anesthetizing action thereof or even decompose the anesthetic Nm. A chemical action of the wall alloy on the liquid anesthetic Nm may lead to so-called Lewis acids being formed. The formation of these Lewis acids may lead to harmful substances, and especially hydrofluoric acid (HF), being formed. The risk of this undesirable action occurs especially when the liquid anesthetic Nm remains in the anesthetic tank 5 for a relatively long time or when the anesthetic container 8 is exposed to a relatively high ambient temperature of above 35° C.

A coating 25, which is arranged between the wall 24 and the liquid anesthetic Nm in the anesthetic tank 5, is applied to the inside of the wall 24 of the anesthetic tank 5, including of the cover 10. The coating 25 covers the entire inside of the wall 24 and largely prevents the liquid anesthetic Nm from coming into contact with the wall 24. In the exemplary embodiment, a coating 27 that covers the entire inside of the wall 26 is applied to the inside of the wall 26 of the neck 7. The coating 25, 27 preferably forms a continuous coating for the entire inner wall of the anesthetic container 8. Two possible exceptions: The inner surface of the closure 23 and the inner surface of the inspection glass 30 are free from this coating 25, 27.

It is also possible that only the inner surface of the anesthetic tank 5 is provided with the coating 25 according to the present invention and the inner surface of the neck 7 is not coated at all or has a different coating. Especially when the neck 7 is arranged in or close to the cover 10, the neck 7 comes into contact with a liquid anesthetic Nm for a shorter time, for example, during the refilling, than the tub of the anesthetic tank 5.

The coating 25, 27 ideally completely prevents a liquid anesthetic in the anesthetic container 8 from coming into contact with the wall 24, 26. In practice, the coating 25, 27 does not cover the wall 24, 26 completely and in a gap-free manner, so that a contact occurs between the wall 24, 26 and the liquid anesthetic Nm in spite of the coating 25, 27. One possible cause are pores in the wall 24, 26, the maximum diameter of which is greater than the layer thickness of the coating 25, 27. In many cases, however, it is possible to achieve that the size of this remaining contact surface is at most 5%, in some cases even at most 1%, of the entire area of the wall 24, 26.

The layer thickness of the coating 25, 27 is in a range of 0.5 µm to 80 µm, preferably in a range of 10 µm to 20 µm. The coating 25, 27 especially preferably has a uniform layer thickness of 15 µm ± 2 µm.

An alloy comprising nickel (Ni) and phosphorus (P) and optionally additional components is used as the material for the coating 25, 27 according to the present invention. This alloy is sufficiently chemically resistant to a liquid anesthetic in the anesthetic container 8 and does not exert an undesirable action on the liquid anesthetic Nm. The phosphorus content in this alloy is at least 3 wt.% and at most 15 wt.%. The phosphorus portion is preferably at least 10 wt.% and at most 13 wt.%. Such a coating is also called nickel phosphorus (NP) or even “chemical nickel.” Thanks to the phosphorus portion, the coating 25, 27 is relatively resistant to wear and corrosion.

In case the phosphorus portion is above 10 wt.%, the coating has, moreover, a fully amorphous structure. Therefore, the risk that inhomogeneities such as grain boundaries or separated phases will develop in the coating 25 is relatively low. Furthermore, the risk that crystals, which may lead to an uneven surface of the coating 25, will be formed during the manufacture of the coating 25, 27 is low.

Different alternatives for manufacturing an anesthetic container 8 according to the present invention will be described below. In all these alternatives, first a part is manufactured, which comprises the two walls 24, 26 and the optional inspection window 30, but no coating according to the present invention on the inner surface.

It is possible to spray the coating 25, 27 onto the inner wall of the provided part 24, 26, 30. It is also conceivable to fill a suitable liquid into the interior of the provided part 24, 26, 30, to leave this liquid there until the coating 25, 27 has formed, and then pouring out the liquid again.

In a preferred embodiment, the coating 25, 27 is, by contrast, generated in a dipping bath with a liquid comprising nickel and phosphorus. The part having the wall 24, 26 and having the optional inspection window 30 is lowered into this dipping batch, especially preferably such that the part 24, 26, 30 is completely submerged in the liquid. The coating 25, 27 is formed on the wall 24, 26 by a chemical or electrochemical reaction.

If a dipping bath is used, then not only is the inside of the wall 24, 26 coated, but also the outside. Possible holes, bulges, undercuts as well as optional lines are likewise coated in the dipping bath. This coating on the outside increases the chemical and mechanical resistance of the wall 24, 26 in many cases.

The coating process by means of a dipping bath leads to the layer thickness of the coating 25, 27 being relatively consistent over the entire extension of the wall 24, 26. It is possible in many cases to achieve that the thickness of the coating 25, 27 varies spatially by at most ± 5 µm or even only by ± 3 µm. Often, it is possible to achieve that unevennesses in the inner surface of the wall 24, 26 of the anesthetic container 8 are compensated for by the coating 25, 27 and even at least some of the pores are closed.

The coating 25, 27 is preferably applied to the part 24, 26, 30 in a dipping bath by means of a redox reaction or by means of a galvanic process with an electrolyte or by anodization. The part 24, 26, 30 is lowered into the dipping bath. The dipping bath comprises, for example, a tub and provides a liquid that contains nickel and phosphorus. During the redox reaction, which is also known by the name electronic nickel or “chemical nickel,” nickel ions are deposited on the inner surface of the wall 24, 26 by means of a chemical oxidation reaction.

The redox reaction generates the necessary electrons proper. Hence, it is not necessary to apply an electrical voltage. In some cases, it is therefore possible to apply the coating 25, 27 to the wall 24, 26 by means of a chemical reaction in a dipping bath even if the wall 24, 26 is manufactured from a plastic or from another material, which is not electrically conductive, and therefore, a galvanic process is not possible. In addition, no supply with electric energy is needed in some cases.

Two chemical partial reactions, namely
(1) 3 NaH₂PO₂ + 3 H₂O + NiSO₄ ➨ 3 NaH₂PO₃ + H₂SO₄ + 2 H₂ + Ni and
(2) NaH₂PO₂ + Hₙₐₛ ➨ H₂O + NaOH + P
take place during the coating in one embodiment.

The lower the pH value of the electrolyte is in the dipping bath, the slower the partial reaction (1) proceeds and the faster the partial reaction (2) proceeds. The phosphorus portion in the coating 25, 27 can be set by means of a suitable selection of the pH value of the liquid in the dipping bath. A pH value of the liquid provided in the dipping bath is therefore derived and predefined as a function of a desired phosphorus portion in the coating 25, 27 to be manufactured. The residence time of the part with the two walls 24, 26 in the dipping bath determines the obtained layer thickness of the coating 25, 27.

The coating 25, 27 is especially preferably manufactured according to DIN EN ISO 4527. The alloy comprising nickel and phosphorus with a phosphorus portion of preferably at least 10 wt.% leads to a supersaturated solution of phosphorus in the nickel.

Some process steps, which belong to the manufacturing process, by means of which the anesthetic tank 8 is manufactured, will be explained below as examples.
A part, which comprises the wall 24, 26 and the optional inspection window 30, is first manufactured.
Lubricants and optionally welding burrs are removed from this part 24, 26.
An oxide layer is removed at least once from the two surfaces of the wall 24, 26.
In addition, the part 24, 26, 30 is rinsed at least once.
The two surfaces of the wall 24, 26 are pretreated so that the coating 25, 27 according to the present invention adheres better.
The part with the wall 24, 26 is inserted into a dipping bath, which contains a mixture comprising nickel and phosphorus in the liquid form and has a predefined pH value. An expanding coating 25, 27 is formed in the dipping bath on the two surfaces of the wall 24, 26. The part 24, 26, 30 is left in the dipping bath until the layer thickness of the coating 25, 27 has reached a predefined lower threshold for the layer thickness. For example, it is empirically determined beforehand at what rate the layer thickness of the coating 25, 27 increases in the dipping bath, and a residence time of the part 24, 26 in the dipping bath is derived from the empirically determined rate of increase and from the predefined lower threshold.

At least some of the steps, removing lubricants as well as an oxide layer, rinsing the part and pretreating the surfaces, can also be carried out in a respective dipping bath.

A user may visually determine the fill level of the liquid anesthetic Nm in the anesthetic tank 5 from outside. Hence, in the embodiment according to FIG. 3, a transparent inspection glass 30 is inserted into the wall 24 of the anesthetic tank 5. An O ring preferably encloses the inspection glass 30 and seals the gap between the inspection glass 30 and the wall 24. This O ring is manufactured from an elastic material, which is sufficiently chemically resistant to anesthetics. An elastomer, especially preferably the material ethylene propylene diene rubber (EPDM), is preferably used as a material for the O ring.

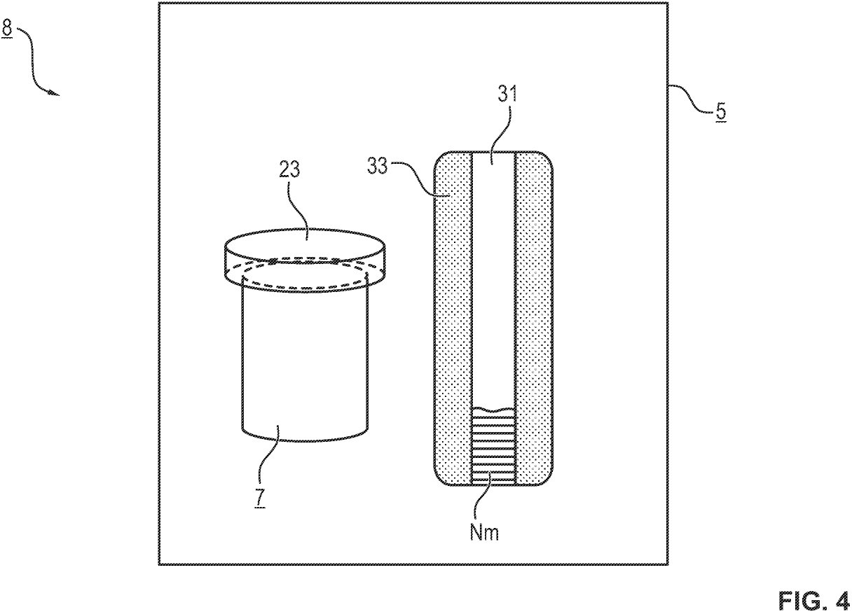

FIG. 4 shows in a front view an alternative embodiment. In the view according to FIG. 4, the neck 7 points toward the viewer. A vertical inspection tube 31 is inserted in a fluid-tight manner into the wall 24 of the anesthetic tank 5. In the example shown, the wall 24 forms an indentation 33 and the inspection tube 31 is enclosed by the indentation 33 such that the inspection tube 33 does not protrude over the wall 24 on the outside. The inspection tube 31 is in a respective fluid connection with the interior of the anesthetic tank 5 both at the top and at the bottom, so that the fill level of the liquid anesthetic Nm in the anesthetic tank 5 is consistent with the fill level in the inspection tube 31 according to the principle of communicating tubes. For example, liquid anesthetic enters into the inspection tube 31 from the bottom during the refilling, and gas may leak out of the inspection tube 31 upwards.

The generic term “visual inspection unit” is used below for the inspection glass 30 from FIG. 3 and the inspection tube 31 from FIG. 4.

In a preferred embodiment, the visual inspection unit 30, 31 is manufactured from a material that has a quartz (silicon dioxide, SiO₂) portion of at least 85 wt.%, preferably a quartz portion of at least 95 wt.%, and especially preferably a quartz portion of at least 99 wt.%.

In addition, the material contains metals, preferably especially aluminum (Al). A material with a quartz portion of at least 85 wt.% is in many cases sufficiently chemically resistant to anesthetics, so that a coating on the inside of the inspection glass 30 is possible, but frequently not necessary.

In an alternative embodiment, the visual inspection unit 30, 31 is manufactured from a material that does not necessarily have a quartz portion of at least 85 wt.%. For example, a borosilicate glass is used as material. Borosilicate glass comprises 70 wt.% to 80 wt.% silicon dioxide (SiO₂), 7 wt.% to 13 wt.% boron trioxide (B₂O₃), 4 wt.% to 8 wt.% alkali oxides, for example, sodium oxide (Na₂O) or potassium oxide (K₂O), as well as optionally additional components.

A transparent coating consisting of a plastic is applied to the inner surface of the visual inspection unit 30, 31 especially in case of a quartz portion below 85 wt.%. This transparent coating on the inner surface prevents an undesirable interaction between the visual inspection unit 30, 31 and the liquid anesthetic Nm in the anesthetic tank 5. The transparent plastic preferably comprises at least one of the following substances:
a Parylene,
a polymer, preferably an epoxy phenolic polymer,
a transparent polytetrafluoroethylene (PTFE),
a polyolefin.

While specific embodiments of the invention have been shown and described in detail to illustrate the application of the principles of the invention, it will be understood that the invention may be embodied otherwise without departing from such principles.

List of Reference Characters

| | |
|---|---|
| 1 | Anesthesia device; it belongs to the ventilation system 200; it comprises the fluid delivery unit 120 |
| 2 | Patient-side coupling unit; it is connected to the patient Pt |
| 3 | Pressure sensor; it measures a value indicative of the pressure in the anesthetic tank 5 |
| 4 | Fill level sensor; it measures a value indicative of the fill level of the liquid anesthetic Nm in the anesthetic tank 5 |
| 5 | Anesthetic tank; it contains the liquid anesthetic Nm; it comprises the wall 24 and the coating 25 |
| 7 | Closable neck for refilling liquid anesthetic Nm into the anesthetic tank 5 |
| 8 | Anesthetic container; it comprises the anesthetic tank 5 and the neck 7 |
| 10 | Cover on the tub of the anesthetic tank 5; it belongs to the wall 24 |
| 11 | Proportional valve in the line 40 |
| 12 | Actuatable injection valve for liquid anesthetic; it acts as a feed device |
| 13 | Vaporizer chamber, in which liquid anesthetic Nm is vaporized or evaporated; it belongs to the anesthetic vaporizer 50 |
| 14 | Temperature sensor; it measures a value indicative of the temperature in the vaporizer chamber 13; it belongs to the anesthetic vaporizer 50 |
| 15 | Pneumatic resistance in the line 41; it can be optionally actuated |
| 16 | Anesthetic heater of the anesthetic vaporizer 50; it heats the vaporizer chamber 13; it belongs to the anesthetic vaporizer 50 |
| 17 | Inlet, via which the carrier gas is fed to the gas mixture generator 100 |
| 18 | Mixing chamber, in which the gaseous anesthetic is mixed with the carrier gas; it belongs to the gas mixer 60 |
| 19 | Outlet, via which the anesthetization gas mixture comprising carrier gas and anesthetic is discharged from the gas mixture generator 100 |
| 20 | Supply port in the wall W for carrier gas |
| 21 | Supply port in the wall W for compressed air |
| 22 | Disposal port in the wall W for receiving excess gas mixture from the ventilation system 200 |
| 23 | Removable closure for the neck 7 |
| 24 | Wall of the anesthetic tank 5; it is provided with the coating 25 on the inner surface |
| 25 | Coating on the inner wall of the wall 24 of the anesthetic tank 5, consisting of nickel with at least 3 wt.% and at most 15 wt.% phosphorus |
| 26 | Wall of the neck 7; it is provided with the coating 27 on the inside |
| 27 | Coating on the inner wall of the neck 7 |
| 30 | Inspection glass for visually determining the fill level in the anesthetic tank 5; it is inserted into the wall 24 in a fluid-tight manner; it is manufactured from quartz glass or borosilicate glass; it optionally comprises an inner coating |
| 31 | Inspection tube for visually determining the fill level in the anesthetic tank 5; it is arranged in the indentation 33; it is connected to the wall 24 in a fluid-tight manner |
| 32 | Refill container for liquid anesthetic Nm; it can be placed on the neck 7 |
| 33 | Indentation in the wall 24; it accommodates the inspection tube 31 |
| 40 | Vaporizer feed line; it leads from the anesthetic tank 5 to the vaporizer chamber 13 |
| 41 | Mixer feed line; it leads from the vaporizer chamber 13 to the mixing tank 18 |
| 49 | Filter |
| 50 | Anesthetic vaporizer; it comprises the vaporizer chamber 13, the anesthetic heater 16 and the temperature sensor 14; it belongs to the anesthetic dispenser 50 |
| 59 | Port of the anesthetic dispenser 100, via which the pressure in the anesthetic tank 5 is controlled |
| 60 | Gas mixer; it comprises the mixing chamber 18, the mixing chamber heater 69 and the temperature sensor 70; it generates an anesthetization gas mixture comprising a carrier gas and gaseous anesthetic; it belongs to the gas mixture generator 100 |
| 66 | Proportional valve, with which the pressure in the anesthetic tank 5 is controlled |
| 69 | Mixing chamber heater; it is capable of heating the gas mixture in the mixing chamber 18; it belongs to the gas mixer 60 |
| 70 | Temperature sensor; it measures a value indicative of the temperature in the mixing chamber 18; it belongs to the gas mixer 60 |
| 80 | Anesthetic dispenser; it comprises the anesthetic container 8, the anesthetic vaporizer 50 and the vaporizer feed line 40; it belongs to the gas mixture generator 100 |
| 100, 100.1, 100.2 | Gas mixture generator; it comprises the anesthetic dispenser 80 and the gas mixer 60 |
| 120 | Fluid delivery unit in the form of a pump; it maintains a stream of gas in the ventilation circuit |
| 130 | Fluid connection between the ventilation system 200 and the patient-side coupling unit 2 |
| 200 | System for the mechanical ventilation of the patient Pt; it comprises the anesthesia device 1, the pump 120 and the anesthetic dispensers 100.1, 100.2 |
| Nm | Liquid anesthetic in the anesthetic tank 5 |
| Pt | Patient; he is mechanically ventilated; he is connected to the patient-side coupling unit 2 |
| W | Wall; it carries the supply ports 20 and 21 and the port 59 |

What is claimed is:

1. An anesthetic container for use in an anesthetic dispenser, the anesthetic container comprising:
an anesthetic tank for receiving a liquid anesthetic, the anesthetic tank comprising a wall with an inner surface and a coating on the inner surface, the inner surface pointing toward a liquid anesthetic in the anesthetic tank; and
a refill unit for refilling liquid anesthetic into the anesthetic tank,
wherein the coating of the anesthetic tank comprises an alloy with a nickel portion in a range of 80 weight % to 97 weight %, and a phosphorus portion in a range of 3 weight % to 15 weight %.

2. An anesthetic container in accordance with claim 1, wherein the range of the phosphorus portion is 10 weight % to 13 weight %.

3. An anesthetic container in accordance with claim 1, further comprising a visual inspection unit configured to provide a visible fill level of liquid anesthetic in the anesthetic tank from outside the anesthetic tank through the visual inspection unit.

4. An anesthetic container in accordance with claim 3, wherein the visual inspection unit comprises a transparent material including a quartz portion of at least 70 weight %, preferably a quartz portion of at least 80 weight %.

5. An anesthetic container in accordance with claim 3, further comprising a transparent coating comprising a plastic applied to an inner surface of the visual inspection unit.

6. The anesthetic container in accordance with claim 5, wherein the plastic of the transparent coating comprises:
a parylene; or
a polymer; or
a transparent polytetrafluoroethylene; or
a polyolefin; or
any combination of a parylene, and a polymer, and a transparent polytetrafluoroethylene, and a polyolefin.

7. An anesthetic container in accordance with claim 1, wherein the wall of the anesthetic tank comprises at least one metal alloy with an aluminum portion of at least 80 weight %.

8. An anesthetic container in accordance claim 1, wherein the wall of the anesthetic tank comprises plastic, the plastic comprising at least one of:
a polyamide; or
a polyphenylene sulfide; or
a polyether ether ketone.

9. An anesthetic container in accordance with claim 1, wherein the refill unit comprises:
a refill unit wall; and
a coating on an inner surface of the refill unit wall, wherein the refill unit wall is connected in a fluid-tight manner to the wall of the anesthetic tank, and wherein a refill unit wall coating of the wall of the refill unit comprises a same material as or is made of the same material than the coating of the anesthetic tank.

10. An anesthetic container in accordance with claim 3, wherein the visual inspection unit comprises a transparent material including a quartz portion of at least 80 weight %.

11. An anesthetic container in accordance with claim 3, wherein the visual inspection unit comprises a transparent material including a quartz portion of at least 90 weight %.

12. An anesthetic container in accordance with claim 3, wherein the visual inspection unit comprises a transparent material including a quartz portion of at least 99 weight %.

13. An anesthetic container in accordance with claim 1, wherein the wall of the anesthetic tank comprises at least one metal alloy with an aluminum portion of at least 95 weight %.

14. An anesthetic dispenser comprising:
an anesthetic container comprising: an anesthetic tank for receiving a liquid anesthetic, the anesthetic tank comprising a wall with an inner surface and a coating on the inner surface, the inner surface pointing toward a liquid anesthetic in the anesthetic; and a refill unit for refilling liquid anesthetic into the anesthetic tank, wherein the coating of the anesthetic tank comprises an alloy with a nickel portion in a range of 80 weight % to 97 weight %, and a phosphorus portion in a range of 3 weight % to 15 weight %;
a feed device configured to be in a fluid connection with the anesthetic container; and
an anesthetic vaporizer, wherein the feed device is configured to feed liquid anesthetic from the anesthetic container into the anesthetic vaporizer, and wherein the anesthetic vaporizer is configured to generate gaseous anesthetic with the use of the liquid anesthetic fed in by the feed unit.

15. An anesthetic dispenser in accordance with claim 14, in combination with a gas mixer to form a gas mixture generator, wherein the gas mixer is configured to be in a fluid connection with the anesthetic dispenser, wherein the gas mixer is configured to generate a gas mixture comprising oxygen and at least one gaseous anesthetic, wherein the gaseous anesthetic is generated by the anesthetic dispenser.

16. An anesthetic dispenser in combination with a gas mixer to form a gas mixture generator according to claim 15, in combination with a fluid delivery unit to form a ventilation system for the mechanical ventilation of a patient, wherein the patient is connected or connectable to a patient-side coupling unit, wherein a fluid connection is established or establishable between the ventilation system and the patient-side coupling unit, wherein the fluid delivery unit is configured to deliver the gas mixture through the fluid connection to the patient-side coupling unit.

17. A process for manufacturing an anesthetic container, the anesthetic container comprising: an anesthetic tank for receiving a liquid anesthetic, the anesthetic tank comprising a wall with an inner surface and a coating on the inner surface, the inner surface pointing toward a liquid anesthetic in the anesthetic tank; and a refill unit for refilling liquid anesthetic into the anesthetic tank, wherein the coating of the anesthetic tank comprises an alloy with a nickel portion in a range of 80 weight % to 97 weight %, and a phosphorus portion in a range of 3 weight % to 15 weight %, wherein a lower threshold is predefined for a layer thickness of the coating on the inner surface of the anesthetic tank and a pH value is predefined as a function of the predefined range for the phosphorus portion in the coating on the inner surface, and wherein the process comprises the steps of:
manufacturing the wall of the anesthetic tank;
providing a dipping bath with a liquid containing nickel and phosphorus, wherein the liquid has the predefined pH value,
moving the wall in to a dipping bath;
wherein the liquid in the dipping bath encloses the wall after the movement; and
leaving the wall in the dipping bath, so that a coating is formed on both sides of the wall and leaving the wall in the dipping bath until at least the coating on the inner surface of the wall reaches a wall thickness that is greater than or equal to the predefined lower threshold for the layer thickness.

18. A process in accordance with claim 17, wherein an electroless nickel plating process is used during the step of leaving the wall in the dipping bath, so that the two coatings are formed.

19. A process in accordance with claim 17 wherein:
the refill unit comprises: a refill unit wall; and a coating on an inner surface of the refill unit wall, wherein a refill unit wall coating of the wall of the refill unit comprises a same material as or is made of the same material than the coating of the anesthetic tank; and
a part comprising the wall of the anesthetic tank and the wall of the refill unit is manufactured such that the two walls are connected to one another in a fluid-tight manner, the part is moved into the dipping bath, and the part is left in the dipping bath until at least the coating on the inner surface of the two walls has reached a wall thickness that is greater than or equal to the predefined lower threshold for the layer thickness.

20. A process for manufacturing an anesthetic container comprising the process steps of:
providing an anesthetic container comprising an anesthetic tank for receiving a liquid anesthetic, the anesthetic tank comprising a wall with an inner surface and a coating on the inner surface pointing toward a liquid anesthetic space; and a refill unit for refilling liquid anesthetic into the anesthetic tank, wherein the coating of the anesthetic tank comprises an alloy with a nickel content in a range of 80 weight % to 97 weight %, and a phosphorus content in a range of 3 weight % to 15 weight %.
